(19) United States
(12) Patent Application Publication
(10) Pub. No.: US 2025/0017839 A1
(43) Pub. Date: Jan. 16, 2025

(54) COMPOSITION FOR FAT FORMATION INHIBITION AND BODY FAT REDUCTION, CONTAINING HYDRANGENOL AS ACTIVE INGREDIENT

(71) Applicant: COXMAX BIO CO., LTD., Jecheon-Si (KR)

(72) Inventors: Sun Hee LEE, Seongnam (KR); Yu Kyong Shin, Yongin (KR); Hye Shin Ahn, Bucheon (KR); Kyung Tae Lee, Seoul (KR); Kyung Sook Chung, Goyang (KR); Ji Sun Shin, Seoul (KR); Hee Soo Han, Suwon (KR)

(73) Assignee: COXMAX BIO CO., LTD., Jecheon-Si (KR)

(21) Appl. No.: 18/895,470

(22) Filed: Sep. 25, 2024

Related U.S. Application Data
(62) Division of application No. 17/266,211, filed on Feb. 5, 2021.

(30) Foreign Application Priority Data
Feb. 11, 2019 (KR) 10-2019-0015639

(51) Int. Cl. A61K 8/49; A23L 33/10; A61K 36/185; A61Q 19/06

(52) U.S. Cl. CPC A61K 8/498; A23L 33/10; A61K 36/185; A61Q 19/06

(57) ABSTRACT

Provided is a composition for inhibiting fat formation and reducing body fat, the composition including hydrangenol as an active ingredient.

The composition of the present disclosure reduces fat accumulation in adipocytes, reduces phosphorylation of mammalian target of rapamycin (mTOR), and increases phosphorylation of forkhead box O1 (FoxO1), and finally, leading to reduction of an expression level of peroxisome proliferator-activated receptor gamma γ (PPAR), and as a result, the composition inhibits formation of triglyceride in adipocytes to exhibit an anti-obesity effect. Accordingly, the composition including hydrangenol disclosed herein as an active ingredient may be usefully applied to the fields of health functional foods or cosmetics for inhibiting fat formation.

### TECHNICAL FIELD

The present disclosure relates to a composition for inhibiting obesity and reducing body fat by reducing fat accumulation in adipocytes, and more specifically, a composition for inhibiting fat formation caused by exposure to excess nutrients and reducing body fat, the composition including hydrangenol which reduces an expression level of peroxisome proliferator-activated receptor gamma γ (PPAR) and reduces adipogenic factors, and thus inhibits accumulation and secretion of triglyceride.

### BACKGROUND ART

Obesity is a very serious disease that increases rapidly around the world, but there is no medically effective treatment. The World Health Organization (WHO) decided that body mass index (BMI) of 30 or higher is obese. Research results have been published that, in 2014, 13% of the world's population are obese, and 2.2 billion out of 7.4 billion people have health problems related to overweight or obesity.

Obesity refers to a condition of excessive accumulation of body fat due to genetic or lifestyle causes. Obesity causes diseases such as adult diseases, chronic degenerative diseases, etc., and overweight and obesity will account for 2% to 7% of total health expenditure in developed countries. Social disorders caused by obesity and secondary complications caused by excessive fat accumulation, such as hyperlipidemia, hypertension, arteriosclerosis, diabetes, fatty liver, etc., become problems.

The world's obesity population is rapidly growing, despite the WHO's 2013 declaration of obesity as a new epidemic of the 21st century and the world's declaration of war on obesity. Since inducing individual behavior change to prevent obesity cannot be a perfect alternative for individuals who lead busy lives in complex industrial societies, a new problem is emerging in the field of anti-obesity and obesity therapy. Further, despite the development of basic medical and biological research on obesity, obesity is actually increasing, which indicates that there is a gap between these two factors. Therefore, it is required to develop and study foods and cosmetics that are more accessible and may achieve long-term anti-obesity effects.

3T3-L1 adipocytes (pre-adipocytes) activate many transcription factors during differentiation and accumulates fat. Representative differentiation factors are CCAAT/enhancer binding protein alpha (C/EBPa) and peroxisome proliferator activated receptor gamma (PPARγ) induced thereby. By expression of the upstream factors, many downstream proteins are synthesized, and these proteins promote synthesis and storage of triglyceride, which is harmful to our body, and thus a lot of fats are accumulated in cells, leading to obesity.

Hydrangenol is a representative component found in hydrangea (Japanese Patent Publication No. JP-0029934), and its molecular weight is 256.25 g/mol and its IUPAC name is 8-hydroxy-3-(4-hydroxyphenyl)-3,4-dihydroisochromen-1-one. Further, derivatives thereof include (−)-hydrangenol 4′-O-glucoside, and (+)-hydrangenol 4′-O-glucoside. It is known to have functions of skin whitening (Japanese Patent Publication No. JP-0007546) and anti-inflammatory effect (Kim, H. J, et al., Hydrangenol inhibits lipopolysaccharide-induced nitric oxide production in BV2 microglial cells by suppressing the NF-κB pathway and activating the Nrf2-mediated HO-1 pathway, International immunopharmacology v. 35, pp. 61-69).

However, no studies have been conducted on a mechanism of inhibition of lipid accumulation of a composition for inhibiting fat formation and reducing body fat, the composition including hydrangenol as an active ingredient. Therefore, the present inventors studied a direct efficacy of the substance on inhibition of fat accumulation.

Accordingly, the present inventors have made efforts to overcome the problems of the prior art, and as a result, they found that the hydrangenol substance reduces adipogenic factors such as PPARγ to inhibit accumulation and secretion of triglyceride, leading to inhibition of fat formation and reduction of body fat, thereby completing the present disclosure.

### DESCRIPTION OF EMBODIMENTS

### Technical Problem

An aspect provides a health functional food composition for preventing or improving obesity, the health functional food composition including hydrangenol or a pharmaceutically acceptable salt thereof as an active ingredient.

Another aspect provides a pharmaceutical composition for preventing or treating obesity, the pharmaceutical composition including hydrangenol or a pharmaceutically acceptable salt thereof as an active ingredient.

Still another aspect provides a health functional food composition for preventing or improving obesity, the health functional food composition including a hydrangenol-containing hydrangea extract as an active ingredient.

Still another aspect provides a pharmaceutical composition for preventing or treating obesity, the pharmaceutical composition including a hydrangenol-containing hydrangea extract as an active ingredient.

Still another aspect provides a health functional food composition for preventing or improving a metabolic disease, the health functional food composition including hydrangenol or a pharmaceutically acceptable salt thereof as an active ingredient.

Still another aspect provides a pharmaceutical composition for preventing or treating a metabolic disease, the pharmaceutical composition including hydrangenol or a pharmaceutically acceptable salt thereof as an active ingredient.

Still another aspect provides a health functional food composition for preventing or improving a metabolic disease, the health functional food composition including a hydrangenol-containing hydrangea extract as an active ingredient.

Still another aspect provides a pharmaceutical composition for preventing or treating a metabolic disease, the pharmaceutical composition including a hydrangenol-containing hydrangea extract as an active ingredient.

Still another aspect provides a method of preventing, improving, or treating obesity or a metabolic disease, the method including administering an effective amount of hydrangenol or a pharmaceutically acceptable salt thereof to an individual in need thereof.

Still another aspect provides a method of preventing, improving, or treating obesity or a metabolic disease, the method including administering a hydrangenol-containing hydrangea extract to an individual in need thereof.

Still another aspect provides use of hydrangenol or a pharmaceutically acceptable salt thereof in preparing a composition for preventing, improving, or treating obesity or a metabolic disease.

Still another aspect provides use of a hydrangenol-containing hydrangea extract in preparing a composition for preventing, improving, or treating obesity or a metabolic disease.

### Solution to Problem

An aspect provides a health functional food composition for preventing or improving obesity, the health functional food composition including hydrangenol or a pharmaceutically acceptable salt thereof as an active ingredient.

Generally, even though a person is heavy, he is not probably obese when he has a lot of muscles. Therefore, a state in which adipose tissues are excessive in the body is referred to as “obesity”. The term “obesity” refers to a condition in which body fat is excessive, and clinically, the body mass index is 25 in Korea and 30 or more according to the World Health Organization (WHO). In general, obesity means the body weight that is greater than the normal value. However, even though a person is overweight, obesity is diagnosed only when the proportion of body fat among the components of the body is high, and obesity refers to a disease that occurs in both adults and children. Such obesity not only increases body weight, but also may cause overeating, heavy drinking and bulimia, obesity-related diseases, such as hypertension, diabetes, increased plasma insulin level, insulin resistance, hyperlipidemia, metabolic syndrome, insulin resistance syndrome, obesity-related gastroesophageal reflux, arteriosclerosis, hypercholesterolemia, hyperuricemia, lower back pain, cardiac and left ventricular hypertrophy, lipodystrophy, nonalcoholic steatohepatitis, cardiovascular disease, or polycystic ovary syndrome. Therefore, when the composition according to the present disclosure is used, prevention or treatment of not only obesity, but also obesity-related diseases may be simultaneously achieved, and treatment targets for obesity-related diseases include those who want to lose weight.

The term “prevention” refers to a method of partially or completely delaying or preventing onset or recurrence of a disease, a disorder, or accompanying symptoms thereof, a method of preventing the acquisition or reacquisition of a disease or a disorder, or a method of reducing the risk of acquiring a disease or a disorder. For example, the prevention refers to any action that inhibits or delays occurrence of obesity, or obesity-related diseases, disorders, or symptoms by administering the composition according to the present disclosure.

The term “improvement” may refer to any action that at least reduces parameters associated with alleviation or treatment of a condition, e.g., a degree of a symptom.

The term “health functional food” refers to a food prepared and processed, for the maintenance of health, by using a specific ingredient as a raw material or by extracting, concentrating, refining, or mixing a specific ingredient contained in the raw material of the food. The health functional food refers to a food that is designed or processed to sufficiently exert a biological control function such as bio-defense, regulation of biological rhythm, prevention and recovery of a disease due to such components. The composition for health food may perform functions related to the prevention of obesity and the recovery of obesity-related diseases.

The “health functional food composition” may be formulated into a common health functional food formulation known in the art. For example, the composition may be prepared in general formulations such as powders, granules, tablets, pills, capsules, suspensions, emulsions, syrups, infusions, liquids, extracts, etc., and prepared in any health food such as meat, sausages, bread, chocolate, candy, snacks, confectionery, pizza, ramen, other noodles, gums, jelly, dairy products including ice cream, various soups, beverages, teas, drinks, alcoholic beverages, and vitamin complexes. For the formulation of the health foods, a carrier or an additive acceptable for use in food may be used, and any carrier or additive known in the art to be applicable in the preparation of a formulation may be used. The additive may include various nutrients, vitamins, electrolytes, flavoring agents, coloring agents, pectic acid and salts thereof, alginic acid and salts thereof, organic acids, protective colloidal thickeners, pH adjusters, stabilizers, preservatives, glycerin, alcohol, a carbonation agent used in carbonated beverages, etc. In addition, fruit pulp for preparing a natural fruit juice, a fruit juice beverage, or a vegetable beverage may be included. These components may be used independently or in combination. Proportions of the additives may be 0.001% by weight to 5% by weight, specifically, 0.01% by weight to 3% by weight, based on the total weight of the composition.

The content of the hydrangenol or pharmaceutically acceptable salt thereof in the health food composition may be appropriately determined according to the purpose of use (prevention or improvement). In general, it may be included in an amount of 0.01% by weight to 15% by weight, based on the total weight of the food, and when prepared into a beverage, it may be included in an amount of 0.02 g to 10 g, and specifically, 0.3 g to 1 g, based on 100 mL.

The beverage may further include ingredients other than the composition, and may further include various flavoring agents or natural carbohydrates that are commonly used in beverages. The natural carbohydrates may include common sugars such as monosaccharides (e.g., glucose, fructose, etc.), disaccharides (e.g., maltose, sucrose, etc.), polysaccharides (e.g., dextrin, cyclodextrin, etc.), and sugar alcohols such as xylitol, sorbitol, erythritol, etc. In addition, the flavoring agents may include natural flavoring agents (e.g., taumatin, stevia extract, etc.) and synthetic flavoring agents (e.g., saccharin, aspartame, etc.). A proportion of the natural carbohydrate may be generally about 1 g to about 20 g, and specifically, about 5 g to about 12 g per 100 mL of the beverage.

In one specific embodiment, the hydrangenol may be represented by the following Formula 1:

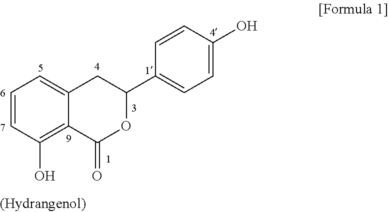 [SMILES: O=C1OC(c2ccc(O)cc2)Cc2cccc(O)c21]

In one specific embodiment, the hydrangenol or the pharmaceutically acceptable salt thereof may inhibit fat formation or may reduce body fat.

In one specific embodiment, the hydrangenol may be isolated from a hydrangea extract.

Another aspect provides a pharmaceutical composition for preventing or treating obesity, the pharmaceutical composition including hydrangenol or a pharmaceutically acceptable salt thereof as an active ingredient

In one specific embodiment, the hydrangenol or the pharmaceutically acceptable salt thereof may inhibit fat formation or may reduce body fat.

In one specific embodiment, the hydrangenol may be isolated from a hydrangea extract.

The term “pharmaceutical composition” may refer to a molecule or a compound that imparts several beneficial effects when administrated to a subject. The beneficial effect may include enabling of diagnostic decisions; improvement of a disease, symptom, disorder, or pathological condition; reduction or prevention of incidence of a disease, symptom, disorder, or illness; and general response to a disease, symptom, disorder, or pathological condition.

The pharmaceutical composition may be parenterally administered during clinical administration, and may be used in the form of a general medicine formulation. Parenteral administration may refer to administration through a route other than oral administration, such as rectal, intravenous, intraperitoneal, intramuscular, intra-arterial, transdermal, intranasal, inhalation, ocular, and subcutaneous administration. When the pharmaceutical composition of the present disclosure is used as a medicine, it may further include one or more active ingredients exhibiting the same or similar function.

Types of pharmaceutically active ingredients that are able to deliver the active ingredient into an individual may include anticancer agents, contrast agents (dyes), hormones, anti-hormones, vitamins, calcium agents, inorganic agents, saccharides, organic acid preparations, protein amino acid preparations, detoxification agents, enzymes, metabolic preparations, combination preparations for diabetes, tissue growth stimulants, chlorophyll agents, pigment preparations, anti-tumor agents, therapeutic agents for tumor, radiopharmaceuticals, tissue cell diagnostic agents, tissue cell therapeutic agents, antibiotic preparations, antiviral agents, complex antibiotics, chemotherapy, vaccines, toxins, toxoids, antitoxins, leptospira serum, blood products, biological agents, analgesics, immunogenic molecules, antihistamines, anti-allergy medications, non-specific immunogenic agents, anesthetics, stimulants, psychotropic agents, small molecule compounds, nucleic acids, aptamers, antisense nucleic acids, oligonucleotides, peptides, siRNAs, micro RNAs, etc.

When the above pharmaceutical composition is formulated, it is prepared using diluents or excipients, such as fillers, extenders, binders, wetting agents, disintegrants, surfactants, etc., which are commonly used. Formulations for parenteral administration include sterilized aqueous solutions, non-aqueous solvents, suspensions, emulsions, freeze-dried formulations, and suppositories. The non-aqueous solvents or suspending media may include propylene glycol, polyethylene glycol, vegetable oils such as olive oil, injectable esters such as ethyl oleate, etc. As a suppository base, witepsol, macrogol, tween 61, cacao butter, lauric butter, glycerogelatin, etc. may be used.

Further, the pharmaceutical composition may be used after being mixed with a variety of pharmaceutically acceptable carriers such as physiological saline or organic solvents. To increase stability or absorption, carbohydrates such as glucose, sucrose, or dextran, antioxidants such as ascorbic acid or glutathione, chelating agents, low molecular weight proteins, or other stabilizers may be used.

Still aspect provides a health functional food composition for preventing or improving obesity, the health functional food composition including a hydrangenol-containing hydrangea extract as an active ingredient.

In one specific embodiment, the hydrangea extract may be extracted by using water, C1 to C4 alcohol, or a mixed solvent thereof.

In one specific embodiment, the hydrangea extract may be a hot water extract.

The extract may be extracted by a hydrophilic solvent, for example, alcohol, water, or a combination thereof. The alcohol may be a compound having one or more —OH groups of C1 to C10. The alcohol may be C1 to C6 alcohol or C3 to C6 polyhydric alcohol. The alcohol may be methanol, ethanol, n-propanol, isopropanol, n-butanol, sec-butanol, isobutanol, tert-butanol, n-pentanol, n-hexanol, or a mixture thereof. The solvent may be, for example, a mixture of water and alcohol, i.e., an aqueous alcohol solution. The alcohol concentration of the aqueous alcohol solution may be 1% (w/w) to 100% (w/w), for example, 1% (w/w) to 99.5% (w/w), 10% (w/w) to 100% (w/w), 20 to 100% (w/w), 30% (w/w) to 100% (w/w), 40% (w/w) to 100% (w/w), 50% (w/w) to 100% (w/w), 60% (w/w) to 100% (w/w), 70% (w/w) to 100% (w/w), 75% (w/w) to 100% (w/w), 60% (w/w) to 90% (w/w), 60% (w/w) to 80% (w/w), 65% (w/w) to 75% (w/w), or 70% (w/w). The aqueous alcohol solution may be an aqueous methanol, ethanol, or butanol solution.

The extract may be extracted by a common method in the art, such as heating extraction, pressurized extraction, ultrasonic extraction, hot water extraction, reflux cooling extraction, subcritical extraction, supercritical extraction, etc.

The extract may be included in an amount of 0.001% by weight to 80% by weight, for example, 0.01% by weight to 60% by weight, 0.01% by weight to 40% by weight, 0.01% by weight to 30% by weight, 0.01% by weight to 20% by weight, 0.01% by weight to 10% by weight, 0.01% by weight to 5% by weight, 0.05% by weight to 60% by weight, 0.05% by weight to 40% by weight, 0.05% by weight to 30% by weight, 0.05% by weight to 20% by weight, 0.05% by weight to 10% by weight, 0.05% by weight to 5% by weight, 0.1% by weight to 60% by weight, 0.1% by weight to 40% by weight, 0.1% by weight to 30% by weight, 0.1% by weight to 20% by weight, 0.1% by weight to 10% by weight, or 0.1% by weight to 5% by weight with respect to the total weight of the composition.

Still another aspect provides a pharmaceutical composition for preventing or treating obesity, the pharmaceutical composition including the hydrangenol-containing hydrangea extract as an active ingredient.

Still another aspect provides a health functional food composition for preventing or improving a metabolic disease, the health functional food composition including hydrangenol or a pharmaceutically acceptable salt thereof as an active ingredient.

The metabolic disease may include, for example, obesity, fatty liver, diabetes, hyperlipidemia, hypertension, hypercholesterolemia, high LDL cholesterol, cardiovascular disease and arteriosclerosis, and coronary artery disease. In one specific embodiment, the metabolic disease may be hyperlipidemia, hypercholesterolemia, diabetes, or dyslipidemia.

Still another aspect provides a pharmaceutical composition for preventing or treating a metabolic disease, the pharmaceutical composition including hydrangenol or a pharmaceutically acceptable salt thereof as an active ingredient.

In one specific embodiment, the metabolic disease may be hyperlipidemia, hypercholesterolemia, diabetes, or dyslipidemia.

Still another aspect provides a health functional food composition for preventing or improving a metabolic disease, the health functional food composition including the hydrangenol-containing hydrangea extract as an active ingredient.

Still another aspect provides a pharmaceutical composition for preventing or treating a metabolic disease, the pharmaceutical composition including the hydrangenol-containing hydrangea extract as an active ingredient.

Still another aspect provides a method of preventing, improving, or treating obesity or a metabolic disease, the method including administering an effective amount of hydrangenol or a pharmaceutically acceptable salt thereof to an individual in need thereof.

The individual may be a mammal. The mammal may be a human, dog, cat, cow, goat, or pig.

The administration may be performed through any general route as long as it may allow to reach a target tissue. For example, the administration may be performed through intraocular administration, intraperitoneal administration, intravenous administration, intramuscular administration, subcutaneous administration, intradermal administration, transdermal patch administration, oral administration, intranasal administration, intrapulmonary administration, and rectal administration. Specifically, the administration may be performed as desired through intraocular administration. The administration may be performed systemically or locally.

As used herein, “treatment” and “treating”, or “alleviating” and “improving” are used interchangeably with each other. These terms refer to a method of obtaining an advantageous or desired result, including, but not limited to, a therapeutic benefit and/or a prophylactic benefit. The therapeutic benefit refers to any therapeutically significant improvement of one or more diseases, disorders, or symptoms under treatment, or effects thereon. In the prophylactic benefit, the composition may be administered to an individual at risk of developing a specific disease, disorder, or symptom, or to an individual reporting one or more physiological symptoms of the disease, even though the disease, disorder, or symptom has not yet appeared.

The term “effective amount” or “therapeutically effective amount” refers to an amount of an agent sufficient to produce an advantageous or desired result. The therapeutically effective amount may vary depending on one or more of a subject and pathological condition to be treated, a subject's body weight and age, severity of the pathological condition, mode of administration, etc., which may be easily determined by those skilled in the art. Further, the term applies to the capacity to provide an image for detection by any of the imaging methods described herein. The specific dosage may vary depending on one or more of a particular agent selected, a dosage regimen that follows, whether or not it is administered in combination with other compounds, time of administration, a tissue being imaged and a body delivery system carrying the same.

The administration of hydrangenol or a pharmaceutically acceptable salt thereof may be performed at a daily dose of 0.1 mg to 1,000 mg, for example, 0.1 mg to 500 mg, 0.1 mg to 100 mg, 0.1 mg to 50 mg, 0.1 mg to 25 mg, 1 mg to 1,000 mg, 1 mg to 500 mg, 1 mg to 100 mg, 1 mg to 50 mg, 1 mg to 25 mg, 5 mg to 1,000 mg, 5 mg to 500 mg, 5 mg to 100 mg, 5 mg to 50 mg, 5 mg to 25 mg, 10 mg to 1,000 mg, 10 mg to 500 mg, 10 mg to 100 mg, 10 mg to 50 mg, or 10 mg to 25 mg per individual. However, the administration dosage may be variously prescribed depending on factors such as a formulation method, an administration method, a patient's age, body weight, sex, pathological condition, diet, time of administration, route of administration, excretion rate, and response sensitivity. Taking into account these factors, the dosage may be appropriately adjusted by those skilled in the art Administration frequency may be once a day, or twice or more a day within the range of clinically acceptable side effects, and the site of administration may be one, two or more sites, and a total of the number of administration may be from 1 day to 30 days per treatment daily or every 2 to 5 days. If necessary, the same treatment may be repeated after an appropriate time period. For animals other than humans, a dosage that is the same as that of per kg in a human, or for example, a dosage that is determined by, for example, conversion based on the volume ratio (e.g., average value) of organs (e.g., heart, etc.) of a target animal and a human, may be administered.

Still another aspect provides a method of preventing, improving, or treating obesity or a metabolic disease, the method including administering the hydrangenol-containing hydrangea extract to an individual in need thereof.

Still another aspect provides use of hydrangenol or a pharmaceutically acceptable salt thereof in preparing a composition for preventing, improving, or treating obesity or a metabolic disease.

Still another aspect provides use of the hydrangenol-containing hydrangea extract in preparing a composition for preventing, improving, or treating obesity or a metabolic disease.

The terms and methods described in the present disclosure are equally applied to respective disclosures.

### Advantageous Effects of Disclosure

As described above, the present disclosure confirmed that hydrangenol is used as an active ingredient to reduce phosphorylation of mammalian target of rapamycin (mTOR) and to increase phosphorylation of forkhead box O1 (FoxO1), and finally, leading to reduction of adipogenic factors such as peroxisome proliferator-activated receptor gamma γ (PPAR), and as a result, accumulation and secretion of triglyceride may be suppressed, thereby inhibiting fat formation caused by exposure to excess nutrients and reducing body fat. Hydrangenol is a substance derived from natural plants and can be usefully used in the fields of health functional foods and cosmetics.

### BRIEF DESCRIPTION OF DRAWINGS

FIG. 1 shows results of HPLC analysis of hydrangenol included in a hot water extract of hydrangea leaves and a hydrangea extract (Hydrangea serrata);

FIG. 2 shows images and graphs showing results of Oil Red-O staining and quantification to examine changes in triglyceride accumulation in adipocytes treated with hydrangenol or the hydrangea extract;

FIG. 3 shows results of Western blotting to examine expression of triglyceride-regulating proteins in adipocytes treated with hydrangenol or the hydrangea extract;

FIG. 4 shows graphs showing changes in the body weight when a hot water extract of hydrangea leaves was administered to mice at the same time with induction of obesity;

FIG. 5 shows graphs showing changes in the body weight when the hot water extract of hydrangea leaves was administered to mice after induction of obesity;

FIG. 6 shows images of a fat distribution and a body fat content which were measured by dual-energy X-ray absorptiometry, when the hot water extract of hydrangea leaves was administered to mice at the same time with induction of obesity in order to examine body fat-reducing effects of the hot water extract of hydrangea leaves;

FIG. 7A shows a graph showing a body fat content when the hot water extract of hydrangea leaves was administered to mice at the same time with induction of obesity; and FIG. 7B shows a graph showing a fat weight when the hot water extract of hydrangea leaves was administered to mice at the same time with induction of obesity;

FIG. 8 shows images of a fat distribution and a body fat content which were measured by dual-energy X-ray absorptiometry, when the hot water extract of hydrangea leaves was administered to mice after induction of obesity in order to examine body fat-reducing effects of the hot water extract of hydrangea leaves;

FIG. 9A shows a graph showing a body fat content when the hot water extract of hydrangea leaves was administered to mice after induction of obesity; and FIG. 9B shows a graph showing a fat weight when the hot water extract of hydrangea leaves was administered to mice after induction of obesity;

FIG. 10 shows microscopic images of adipocytes to examine the effect of reducing the size of lipid droplets by the hot water extract of hydrangea leaves;

FIG. 11A shows a graph showing cholesterol levels when the hot water extract of hydrangea leaves was administered; and FIG. 11B shows a graph showing low density lipoprotein (LDL) levels when the hot water extract of hydrangea leaves was administered;

FIG. 12A shows results of examining expression of p-AMPK protein in the adipose tissue when the hot water extract of hydrangea leaves was administered; and FIG. 12B shows results of examining expression of p-AMPK protein in the liver when the hot water extract of hydrangea leaves was administered;

FIG. 13 shows a graph showing changes in the body weight when hydrangenol was administered to mice in order to examine body weight-reducing effects of hydrangenol;

FIG. 14 shows images of a fat distribution and a body fat content which were measured by dual-energy X-ray absorptiometry, when hydrangenol was administered to mice in order to examine body fat-reducing effects of hydrangenol;

FIG. 15A shows a graph showing a body fat content when hydrangenol was administered; and FIG. 15B shows a graph showing a fat weight when hydrangenol was administered;

FIG. 16 shows microscopic images of adipocytes to examine the effect of reducing the size of lipid droplets by hydrangenol;

FIG. 17A shows a graph showing cholesterol levels when hydrangenol was administered; and FIG. 17B shows a graph showing LDL levels when hydrangenol was administered; and

FIG. 18A shows results of examining expression of p-AMPK protein in the adipose tissue when hydrangenol was administered; and FIG. 18B shows results of examining expression of p-AMPK protein in the liver when hydrangenol was administered.

### MODE OF DISCLOSURE

Hereinafter, the present disclosure will be described in more detail with reference to exemplary embodiments. These exemplary embodiments are only for illustrating the present disclosure, and the scope of the present disclosure is not be construed as being limited to these exemplary embodiments.

### Example 1: Preparation of Hydrangenol-Containing Hydrangea Extract

A hydrangea extract in a composition of the present disclosure was prepared by the following procedure. First, 20 kg of dried hydrangea (Hydrangea serrata) raw material and 300 kg of purified water were put in an extraction tank, and then subjected to reflux extraction at 100° C. for 5 hours. The extracted sample was filtered using a cartridge filter (10 um), and then concentrated under reduced pressure, and water-soluble powder was obtained by spray-drying.

### Example 2: Preparation of Hydrangea Extract-Derived Hydrangenol

The extract powder obtained in Example 1 was subjected to gel filtration using Diaion HP-20. As a developing solvent, solvent fractionation was performed using each 2 L of a mixed solution of 30%, 50%, 70%, 100% methanol and CH₂Cl₂-MeOH (1:1, v/v), and divided into 5 subfractions (392-70EDia1˜5). The subfraction 392-70EDia4 was divided into 7 subfractions (392-70EDia4a-4g) using Sephadex LH-20 and methanol as a developing solvent. Among them, the 392-70EDia4d fraction was recrystallized in methanol to obtain an amorphous compound 1 (hydrangenol) as a single material.

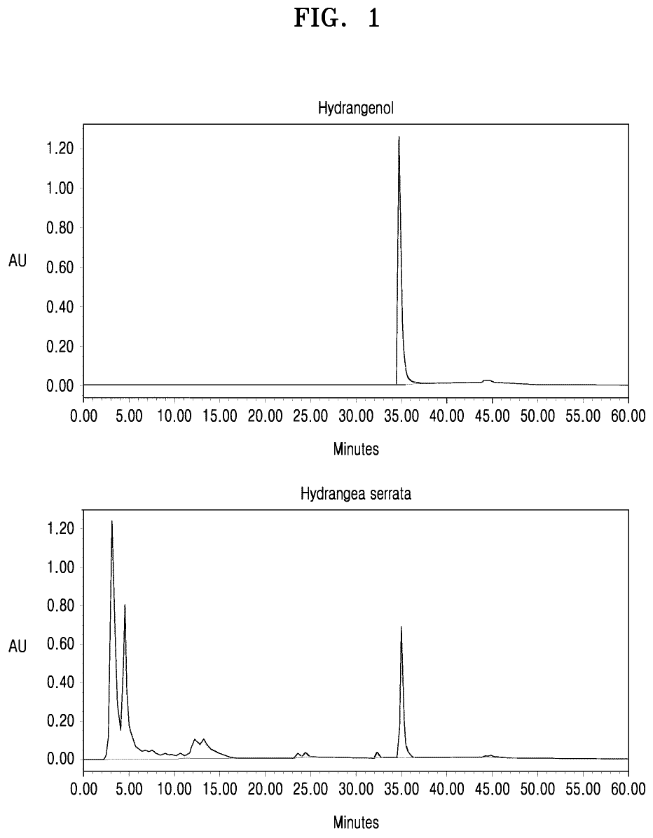

The extract powder obtained in Example 1 and hydrangenol obtained in Example 2 of the present disclosure were analyzed using high-performance liquid chromatography (HPLC) and a UV photometric detector (UV/Vis detector). HPLC instrument was Waters e2695 Series system, Waters 24489 UV/Vis detector (Worcester, MA, USA), and Luna C18(2)(5 μm, 250×4.6 mm, Phenomenex, Torrance, CA, USA) column was used, and all solvents used in the analysis were HPLC grade solvents purchased from J. T. Baker (Phillipsburg, NJ, USA). During the analysis, a temperature of the column was set at 30° C., an injection volume was set at 20 μl, and a measurement wavelength was set at 210 nm. Acetonitrile (ACN) and tertiary distilled water (D.W) were used as a mobile phase, and an ACN-D.W (2:8-10:0, v/v) mixed solution was analyzed for 50 minutes at a rate of 1 ml/min. As an analysis sample, 100 mg of the extract powder obtained in Example 1 was precisely weighed, 10 ml of methanol was added thereto, and then the powder was dissolved in an ultrasonic shaker for 20 minutes, allowed to cool at room temperature, and the supernatant was obtained and then filtered through a 0.45 μm membrane filter for use. 10 mg of the hydrangenol obtained in Example 2 was precisely weighed and 40 ml of methanol was added thereto, and the hydrangenol was dissolved in an ultrasonic shaker for 20 minutes, allowed to cool at room temperature, and methanol was added thereto, and filtered through a 0.45 μm membrane filter for use. For each analysis sample, a chromatogram was extracted at 210 nm, and a peak of hot water extract of hydrangea leaves and a peak of hydrangenol were compared and analyzed (FIG. 1).

The structure of Example 2 was first identified by ESI MS (positive-ion mode), and as a result, m/z=257 [M+H]+ was observed. In 1H-NMR, it was found that the methine proton (H-3) at δH 5.50 at a high magnetic field and the methylene proton (H-4) at δH 3.30 and 3.06 showed vicinal coupling to each other, and the protons were the chemical shift values and were attributable to the C ring. H-2′, 3′ and H-6′, 5′ attributable to the p-substituted benzene ring of the B-ring were ortho-coupled to each other and appeared as doublets (J=8.4 Hz), and peaks of H-2′ and H-6′ and peaks of H-3′ and H-5′ were also ortho-coupled to each other and appeared as doublets, indicating that they had a symmetric structure around the hydroxyl group. In 1,2,3-trisubstituted benzene of A-ring, H-5 and H-7 hydrogens were coupled with H-6 hydrogen, respectively, and H-5 and H-7 hydrogens were ortho-coupled and appeared as doublet, and H-6 proton was ortho- and meta-coupled and appeared as a double of doublets, and all peaks were found to correspond to one hydrogen.

13C-NMR showed a total of 15 peaks including para-substituents. The quaternary carbon at δC 172 was a peak attributable to a carbonyl group which is carbon 1 of the compound, and δC 116.9(C-3′,5′) and 129.6(C-2′,6′) are attributable to para substituents of an aromatic ring. Peaks at δC 36.1 and 83.1 were expected to be attributable to an aliphatic carbon and an oxygenated carbon, respectively. In addition, in DEPT NMR, 7 protonated carbons were identified, and a peak of δC 36.1 was found to be a methylene group attributable to C-4. 2D NMR was analyzed to analyze their exact structures. The exact positions of the peaks were identified from HSQC, and the position at which the substituent was bound was identified from HMBC. That is, the peak of δH 7.26 (2H, d, J=8.4 Hz, H-2′,6′) shows a correlation with C-4 of δC 36.1, and the peaks of δH 3.06 and 3.30 attributable to H-4 show a correlation with the peaks of 83.1 (C-3), 119.8 (C-5), 110.0 (C-9), and 142.2 (C-10). Taken together, hydrangenol was identified.

FIG. 1 shows results of HPLC analysis of hydrangenol included in the hot water extract of hydrangea leaves and the hydrangea extract (Hydrangea serrata).

### Experimental Example 1. Evaluation of Fat Accumulation Inhibition by Oil Red O Staining

In this experiment, to induce adipocyte differentiation, 3T3-L1 cells were dispensed onto a plate and cultured in a 10% BS medium until the cell density reached 100%. At the cell differentiation stage, hydrangenol-containing hydrangea (Hydrangea serrata) (25 ug/ml), hydrangenol (2.5 ug/ml) or a positive control pioglatazone (10 uM) was added to a 10% FBS differentiation medium (5 μg/ml of insulin, 1 μM dexametasone, 0.5 mM 3-isobutyl-1-methylxanthine), respectively. After 10 days of treatment oil Red-O staining and quantitative analysis were performed to determine how much fat accumulation was inhibited. For visual evaluation, images were taken after staining, and then the stained cells were completely dried, dissolved in dimethyl sulfoxide (DMSO), transferred to a 96-well plate, and absorbance at 450 nm was measured.

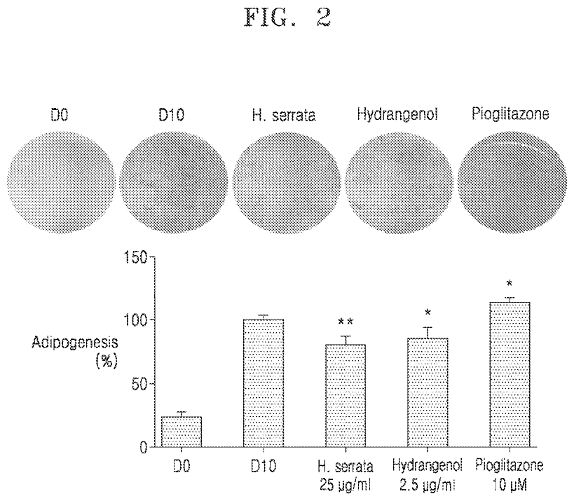

FIG. 2 shows images and graphs showing the results of Oil Red-O staining and quantification to examine changes in triglyceride accumulation in adipocytes treated with hydrangenol or the hydrangea extract.

### Experimental Example 2. Analysis of Expression of Adipocyte Differentiation-Related Proteins During Hydrangenol Treatment

The mechanism of reducing triglyceride in adipocytes was examined for the hydrangenol-containing hydrangea (Hydrangea serrata) and hydrangenol. A pre-adipocyte 3T3-L1 was differentiated for 10 days and treated with hydrangea (Hydrangea serrata) (25 ug/ml) and hydrangenol (2.5 ug/ml) for 24 hours, respectively. Thereafter, the cells were lysed using a modified LIPA buffer, and each 20 ug thereof was used for analysis. p-mTOR(ab109268, Abcam), p-FoxO1(9461S, Cell Signaling), PPARγ(sc-7273, Santa Cruz), and β-actin(A5316, Sigma) primary antibodies were used for analysis, respectively.

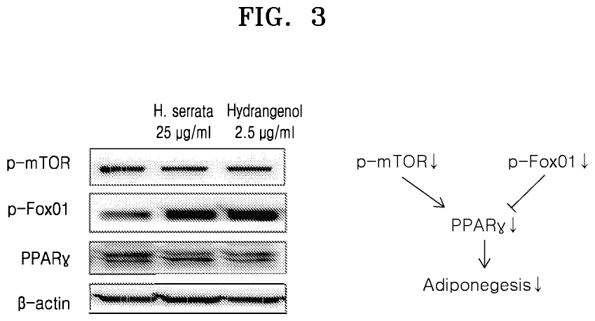

As shown in FIG. 3, hydrangenol-containing hydrangea (Hydrangea serrata) and hydrangenol were found to reduce phosphorylation of mammalian target of rapamycin (mTOR) and to increase phosphorylation of forkhead box O1 (FoxO1), and finally, leading to reduction of an expression level of peroxisome proliferator-activated receptor gamma γ (PPAR), and as a result, triglyceride production in adipocytes was suppressed.

FIG. 3 shows results of Western blotting to examine expression of triglyceride-regulating proteins in adipocytes treated with hydrangenol or the hydrangea extract.

### Experimental Example 3. Analysis of In Vivo Effect of Hot Water Extract of Hydrangea Leaves (WHS)

### 3-1. Mouse and Experiment Design

To analyze in vivo anti-obesity efficacy of WHS, animal models of obesity were first prepared. 8-week-old male C57BL/6N mice (specific-pathogen-free (SPF) grade, 20±2 g, Orient Bio) were set into 7 groups as follows, and 10 mice per each group were tested: as normal control groups, normal mice (con) with no high fat diet and no administration, and obese mice (HFD) with induction of obesity by 30% high fat diet and no administration, and as a positive control group, obese mice with oral administration of orlistat which is an anti-obesity agent. As experimental groups, obese mice with oral administration of WHS of 75 mg/kg, 150 mg/kg, or 300 mg/kg, and normal mice with oral administration of WHS of 300 mg/kg were used. Further, experiments were performed by dividing mice into those which were administered with WHS for 12 weeks at the same time with induction of obesity, and those which were administered with WHS after induction of obesity for 10 weeks. WHS was orally administered for 5 days per week during the administration period. A dark: light cycle was maintained at intervals of 12 hours: 12 hours, and mice were allowed to free access to water.

### 3-2. Analysis of Body Weight- and Fat-Reducing Effects of WHS

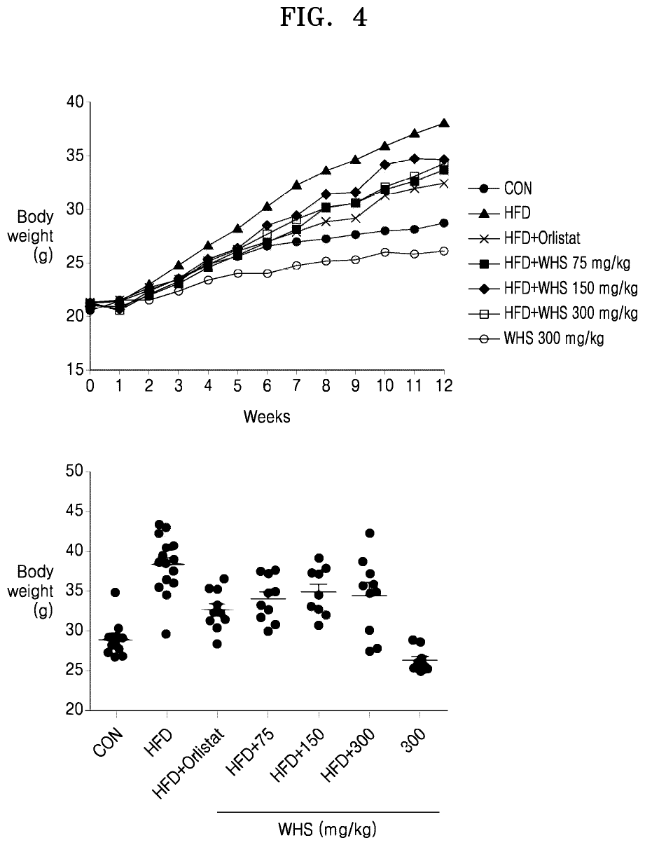

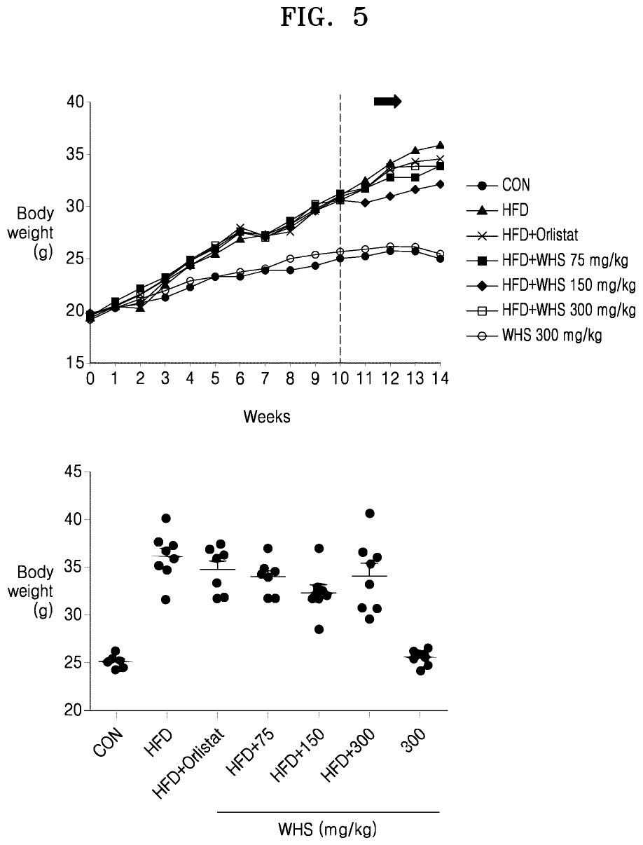

An experiment was performed to analyze whether the body weight and fat of mice decreased when WHS was administered. As a result, when changes in the body weight of the mice according to each experimental group and time were examined, the body weight-reducing effects were observed in the positive control group and the WHS-administered group (FIGS. 4 and 5).

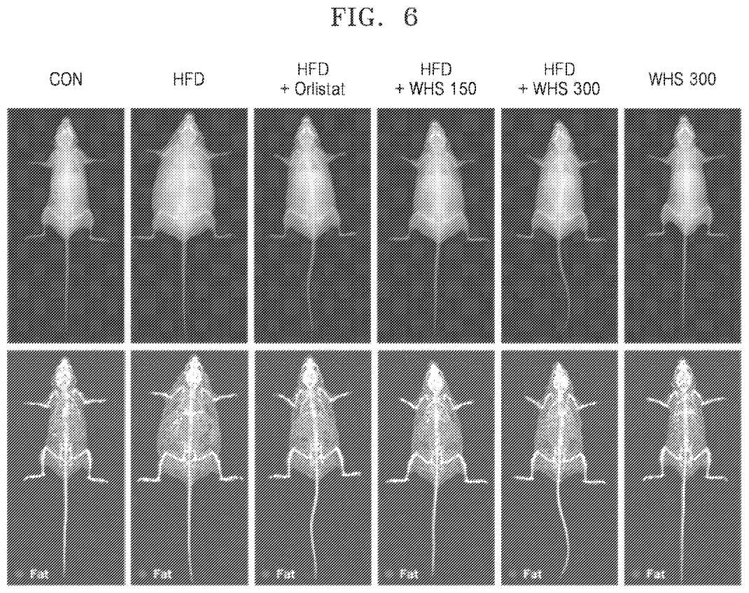

In addition, images of a fat distribution and body fat contents of mice of each experimental group were measured by dual-energy X-ray absorptiometry in the last week of the animal test. Each mice was sacrificed, and a visceral adipose tissue including epididymal fat was separated and a fat was weighed. The experimental results were tested for a significant difference between groups using a t-test in the Sigma plot statistical program (p^{#}<0.05 vs normal control, p*<0.05, p**<0.01, P***<0.001 vs obese group). As a result, it was confirmed that body fat was decreased in the positive control group and the WHS-administered group (FIGS. 6, 7, 8, and 9).

FIG. 4 shows graphs showing changes in the body weight when WHS was administered to mice at the same time with induction of obesity.

FIG. 5 shows graphs showing changes in the body weight when WHS was administered to mice after induction of obesity.

FIG. 6 shows images of a fat distribution and a body fat content which were measured by dual-energy X-ray absorptiometry, when WHS was administered to mice at the same time with induction of obesity, in order to examine body fat-reducing effects of WHS.

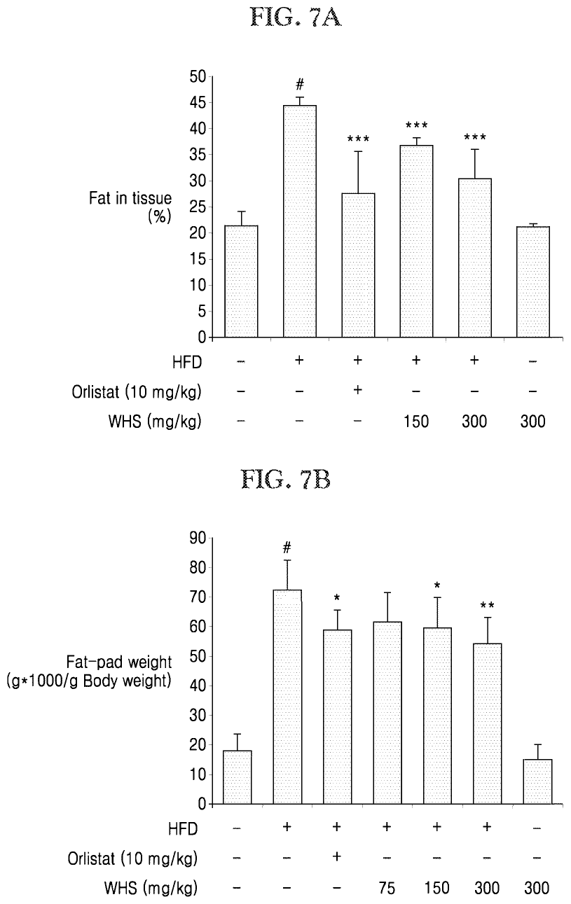

FIG. 7A shows a graph showing a body fat content when WHS was administered to mice at the same time with induction of obesity; and FIG. 7B shows a graph showing a fat weight when WHS was administered to mice at the same time with induction of obesity.

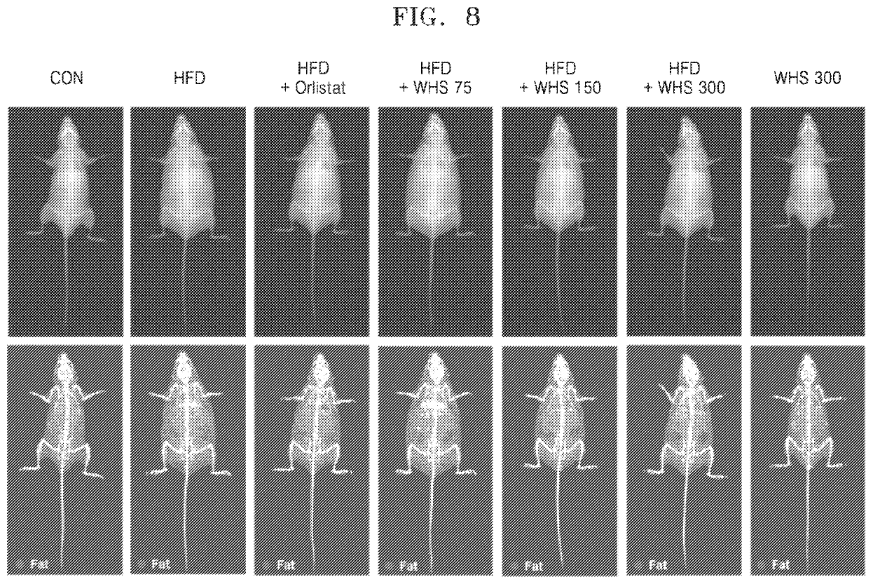

FIG. 8 shows images of a fat distribution and a body fat content which were measured by dual-energy X-ray absorptiometry, when WHS was administered to mice after induction of obesity, in order to examine body fat-reducing effects of WHS.

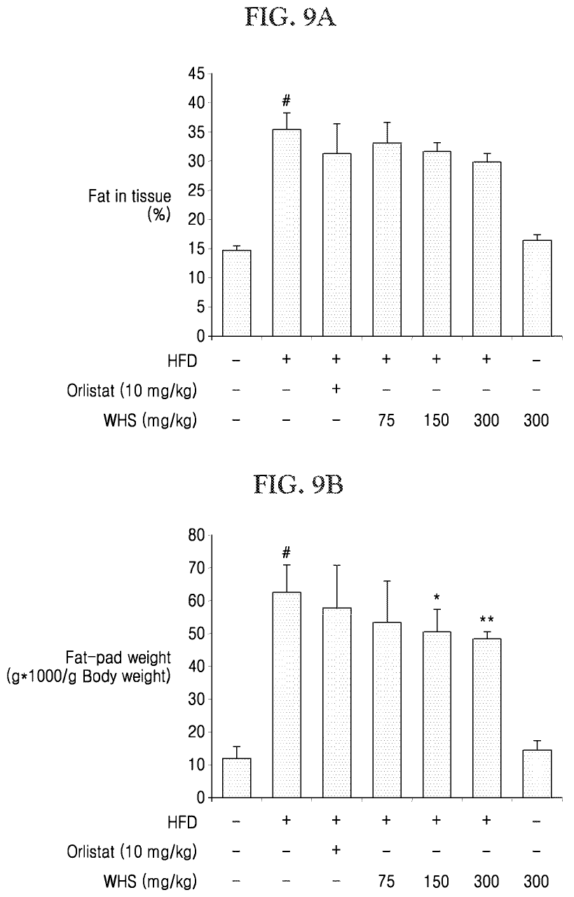

FIG. 9A shows a graph showing a body fat content when WHS was administered to mice after induction of obesity; and FIG. 9B shows a graph showing a fat weight when WHS was administered to mice after induction of obesity.

### 3-3. Analysis of Adipocyte Size-Reducing Effects of WHS

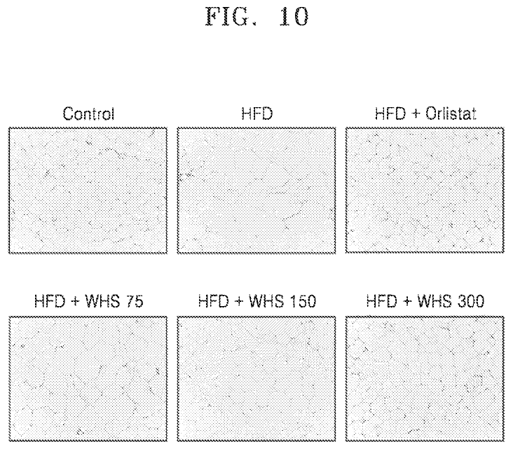

For histological analysis, the epididymal fat tissues of mice were fixed in 4% paraformalin. Dehydration was performed several times through graded alcohol series and washing, and each tissue was embedded in paraffin. Each tissue section was cut at a thickness of 4 μm, and stained with hematoxylin and eosin. In order to examine the size of white adipocytes, the adipocyte area of each section was measured with cellSence software (Olympus Co., USA). As a result, it was confirmed that the adipocyte size was reduced in the group administered with WHS at the same time with induction of obesity (FIG. 10).

FIG. 10 shows microscopic images of adipocytes to examine the effect of reducing the size of lipid droplets by WHS.

### 3-4. Analysis of Effects of WHS on Liver and Kidney

To examine whether WHS induced liver and kidney damage in mice, glutamic oxalacetic transaminase (GOT), glutamic pyruvate transaminase (GPT), and blood urea nitrogen (BUN) of the WHS-administered group with induction of obesity were measured by serum analysis using a biochemical analyzer (AU480 Chemistry Analyzer, Beckman coulter, CA, USA). As a result, as shown in Table 1, there was no significant difference between 7 groups. These results suggest that WHS does not cause liver and kidney damage.

TABLE 1

| | | | HFD + | HFD + WHS | HFD + WHS | HFD + WHS | WHS |
|---|---|---|---|---|---|---|---|
| | CON | HFD | Orlistat | 75 mg/kg | 150 mg/kg | 300 mg/kg | 300 mg/kg |
| GOT μL) | 60.80 ± 15.93 | 64.83 ± 11.32 | 63.00 ± 18.56 | 62.71 ± 17.99 | 60.50 ± 12.82 | 57.78 ± 8.36 | 58.38 ± 15.11 |
| GPT(μL) | 20.43 ± 2.44 | 25.58 ± 4.91 | 17.60 ± 2.17 | 17.40 ± 2.32 | 18.50 ± 2.73 | 16.71 ± 2.93 | 18.89 ± 5.04 |
| BUN(mg/dl) | 22.04 ± 2.58 | 18.75 ± 2.66 | 19.89 ± 3.23 | 18.79 ± 1.12 | 19.47 ± 1.86 | 19.00 ± 2.11 | 25.58 ± 2.65 |

### 3-5. Analysis of Changes of Blood Triglyceride and Blood Cholesterol by WHS

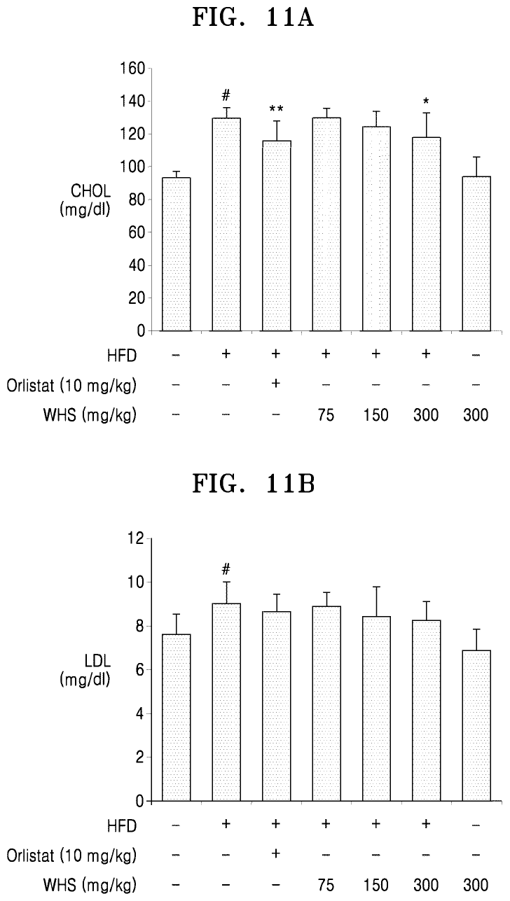

To analyze the effects of WHS on blood triglyceride and blood cholesterol, a hematological-biochemical test of the WHS-administered group with induction of obesity was performed using a biochemical analyzer (AU480 Chemistry Analyzer, Beckman coulter, CA, USA). As a result, as shown in Table 2 and FIG. 11, it was confirmed that the WHS-administered group with induction of obesity showed reduction in the total cholesterol, triglyceride, and LDL levels, but no significant difference in the HDL level. These results indicate that WHS has a prophylactic effect on obesity by reducing total cholesterol, LDL and triglyceride without affecting HDL level.

FIG. 11A shows a graph showing cholesterol levels when WHS was administered; and FIG. 11B shows a graph showing low density lipoprotein (LDL) levels when WHS was administered.

TABLE 2

| | | | HFD + | HFD + WHS | HFD + WHS | HFD + WHS | WHS |
|---|---|---|---|---|---|---|---|
| | CON | HFD | Orlistat | 75 mg/kg | 150 mg/kg | 300 mg/kg | 300 mg/kg |
| CHOL(mg/dl) | 93.00 ± 3.80 | 129.75 ± 5.93 | 115.63 ± 11.87 | 129.11 ± 5.49 | 124.22 ± 9.31 | 117.33 ± 15.29 | 93.60 ± 11.78 |
| LDL(mg/dl) | 7.63 ± 0.92 | 9.00 ± 1.00 | 8.60 ± 0.84 | 8.88 ± 0.64 | 8.44 ± 1.33 | 8.20 ± 0.92 | 6.89 ± 0.93 |
| HDL(mg/dl) | 69.82 ± 8.89 | 84.73 ± 4.65 | 83.70 ± 5.96 | 85.20 ± 6.12 | 82.44 ± 8.43 | 75.40 ± 12.20 | 72.11 ± 6.64 |
| TG(mg/dl) | 46.09 ± 7.08 | 57.27 ± 9.55 | 76.40 ± 9.47 | 44.70 ± 6.55 | 76.11 ± 14.49 | 62.70 ± 16.87 | 64.00 ± 14.74 |

### 3-6. Analysis of Expression of Energy Metabolism-Related Proteins by WHS

AMP-activated protein kinase (AMPK) is activated when energy in hepatocytes decreases to maintain energy homeostasis in the liver, thereby inhibiting synthesis of fat and cholesterol, and conversely, promoting fatty acid oxidation. Therefore, to confirm whether administration of WHS increased AMPK expression, the protein expression level of the WHS-administered group with induction of obesity was analyzed.

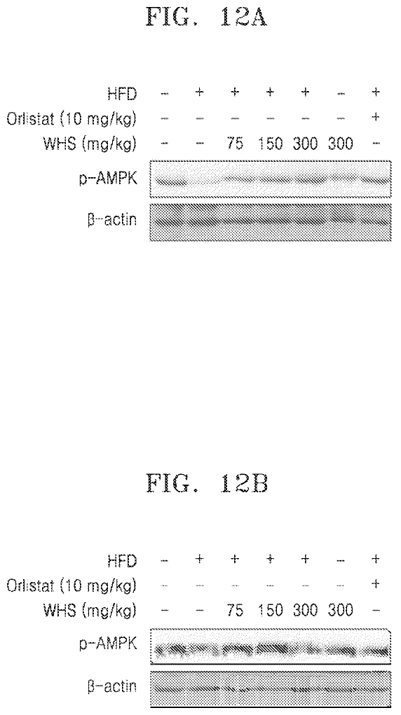

Specifically, adipose and liver tissues were mixed with a protein extraction solution, homogenized using a tissue homogenizer, and then centrifuged at 4° C., 15,000 rpm for 30 minutes to obtain a supernatant, and then a standard curve was created using the Bradford method to quantify protein. 6× sample buffer was added to 30 μg of the protein, followed by heating in a water bath for 5 minutes. Electrophoresis was performed using a 10% SDS-PAGE gel, and immunoblotting was performed on a PVDF membrane for 1 hour and 20 minutes. After blocking for 1 hour with a Tris-buffered saline-Tween 20 (TBST) buffer solution containing 5% (w/v) skim milk, anti-p-AMPK antibody was diluted to 1:1000 and reacted at 4° C. for 18 hours. After washing three times with the TBST buffer solution for 10 minutes, the membrane was reacted with a peroxidase-conjugated secondary antibody for 2 hours at room temperature. After washing three times for 10 minutes with the TBST buffer solution, a hyper film was color-developed and developed using an enhanced chemiluminescence kit (Amersham Life Sciences, Amersham, U.K.) to examine the change of AMPK phosphorylation in each control group and experimental group by Western blotting. As a result, when WHS was administered, the amount of phosphorylated AMPK protein increased (FIG. 12).

These results indicate that WHS has the AMPK phosphorylation-inducing effect which is critical in the anti-obesity effect.

FIG. 12A shows results of examining expression of p-AMPK protein in the adipose tissue when WHS was administered; and FIG. 12B shows results of examining expression of p-AMPK protein in the liver when WHS was administered.

### Experimental Example 4. Analysis of In Vivo Effect of Hydrangenol (HG)

### 4-1 Mouse and Experiment Design

To analyze in vivo anti-obesity efficacy of HG, animal models of obesity were prepared. 8-week-old male C57BL/6N mice (SPF grade, 20±2 g, Orient Bio) were set into 7 groups as follows, and 10 mice per each group were tested: as normal control groups, normal mice (con) with no high fat diet and no administration, and obese mice (HFD) with induction of obesity by 30% high fat diet and no administration, and as a positive control group, obese mice with oral administration of orlistat which is an anti-obesity agent. As experimental groups, obese mice with oral administration of HG of 20 mg/kg, 40 mg/kg, or 80 mg/kg, and normal mice with oral administration of HG of 80 mg/kg were used. Mice were administered with HG at the same time with induction of obesity, and HG was administered for 5 days per week for 12 weeks. A dark: light cycle was maintained at intervals of 12 hours: 12 hours, and mice were allowed to free access to water.

### 4-2 Analysis of Body Weight- and Fat-Reducing Effects of HG

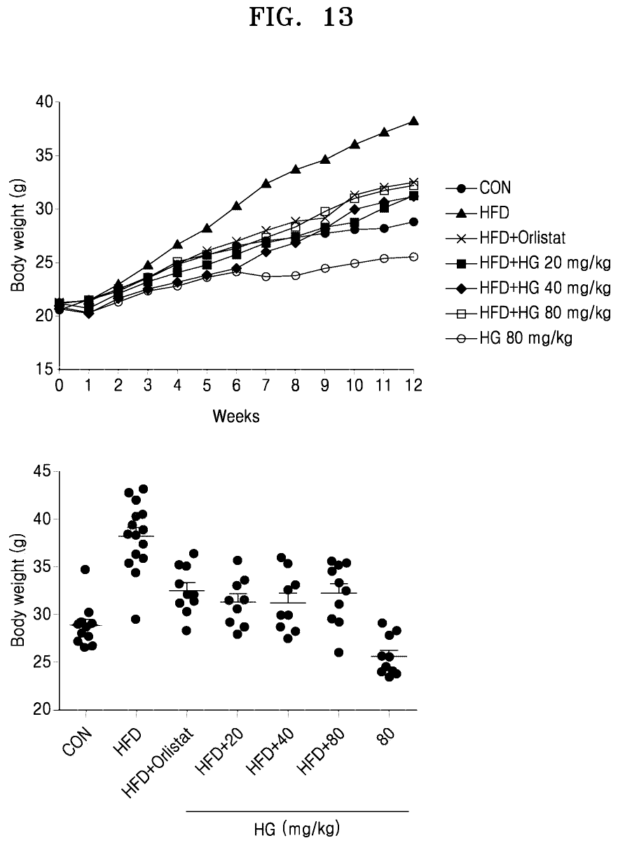

An experiment was performed to analyze whether the body weight and fat of mice decreased when HG was administered. As a result, when changes in the body weight of the mice according to each experimental group and time were examined, the body weight-reducing effects were observed in the positive control group and the HG-administered group (FIG. 13).

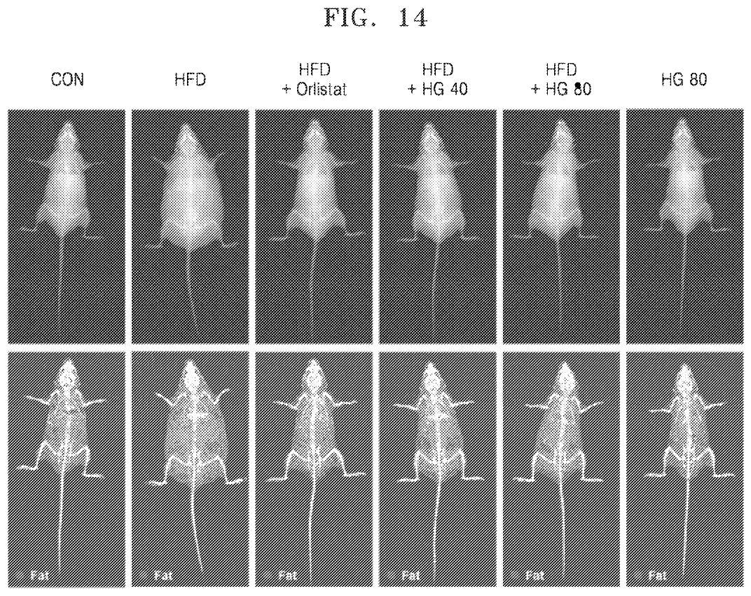

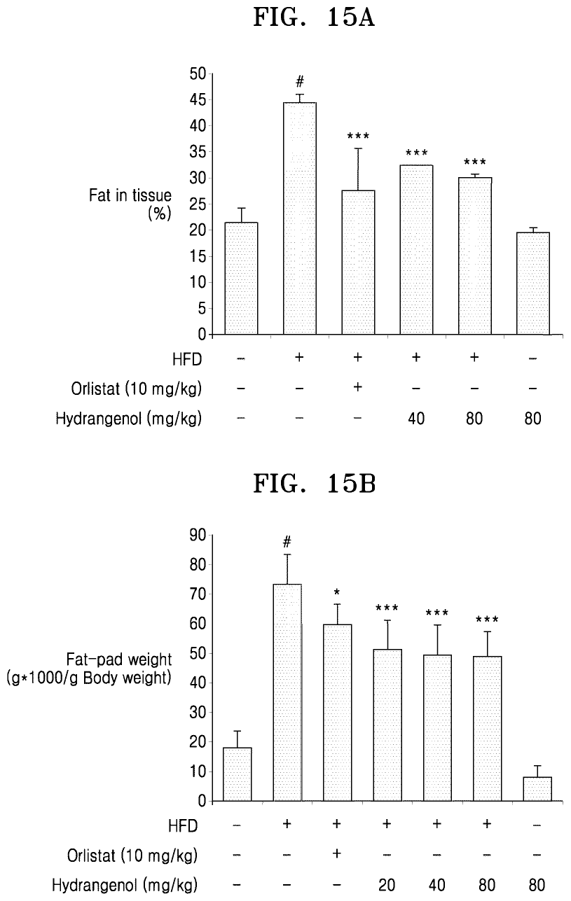

In addition, images of a fat distribution and body fat contents of mice of each experimental group were measured by dual-energy X-ray absorptiometry in the last week of the animal test. Each mice was sacrificed, and a visceral adipose tissue including epididymal fat was separated and a fat was weighed. The experimental results were tested for a significant difference between groups using a t-test in the Sigma plot statistical program (p^{#}<0.05 vs normal control, p*<0.05, p**<0.01, P***<0.001 vs obese group). As a result, it was confirmed that body fat was decreased in the positive control group and the HG-administered group (FIGS. 14 and 15).

FIG. 13 shows a graph showing changes in the body weight when HG was administered to mice in order to examine body weight-reducing effects of HG.

FIG. 14 shows images of a fat distribution and a body fat content which were measured by dual-energy X-ray absorptiometry, when HG was administered to mice in order to examine body fat-reducing effects of HG.

FIG. 15A shows a graph showing a body fat content when HG was administered; and FIG. 15B shows a graph showing a fat weight when HG was administered.

### 4-3 Analysis of Adipocyte Size-Reducing Effects of HG

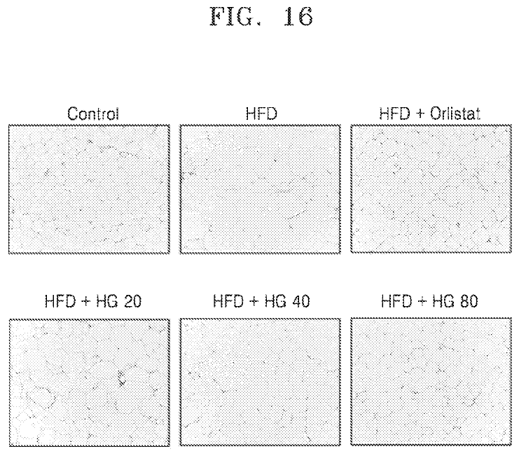

For histological analysis, the epididymal fat tissues of mice were fixed in 4% paraformalin. Dehydration was performed several times through graded alcohol series and washing, and each tissue was embedded in paraffin. Each tissue section was cut at a thickness of 4 μm, and stained with hematoxylin and eosin. In order to examine the size of white adipocytes, the adipocyte area of each section was measured with cellSence software (Olympus Co., USA). As a result, it was confirmed that the adipocyte size was reduced when HG was administered (FIG. 16).

FIG. 16 shows microscopic images of adipocytes to examine the effect of reducing the size of lipid droplets by HG.

### 4-4 Analysis of Effects of HG on Liver and Kidney

To examine whether HG induced liver and kidney damage, glutamic oxalacetic transaminase (GOT), glutamic pyruvate transaminase (GPT), and blood urea nitrogen (BUN) were measured by serum analysis using a biochemical analyzer (AU480 Chemistry Analyzer, Beckman coulter, CA, USA). As a result, as shown in Table 3, there was no significant difference between 7 groups. These results suggest that HG does not cause liver and kidney damage.

TABLE 3

| | | | HFD + | HFD + HG | HFD + HG | HFD + HG | HG |
|---|---|---|---|---|---|---|---|
| | CON | HFD | Orlistat | 20 mg/kg | 40 mg/kg | 80 mg/kg | 80 mg/kg |
| GOT (μL) | 60.80 ± 15.93 | 64.83 ± 11.32 | 63.00 ± 18.56 | 54.88 ± 11.66 | 54.14 ± 10.29 | 53.63 ± 9.91 | 60.14 ± 8.73 |
| GPT(μL) | 20.43 ± 2.44 | 25.58 ± 4.91 | 17.60 ± 2.17 | 16.00 ± 5.03 | 15.71 ± 1.60 | 15.00 ± 2.98 | 16.89 ± 2.62 |
| BUN(mg/dl) | 22.04 ± 2.58 | 18.75 ± 2.66 | 19.89 ± 3.23 | 21.91 ± 3.33 | 22.93 ± 2.74 | 25.39 ± 5.02 | 27.79 ± 4.85 |

### 4-5 Analysis of Changes of Blood Triglyceride and Blood Cholesterol by HG

To analyze the effects of HG on blood triglyceride and blood cholesterol, a hematological-biochemical test was performed using a biochemical analyzer (AU480 Chemistry Analyzer, Beckman coulter, CA, USA).

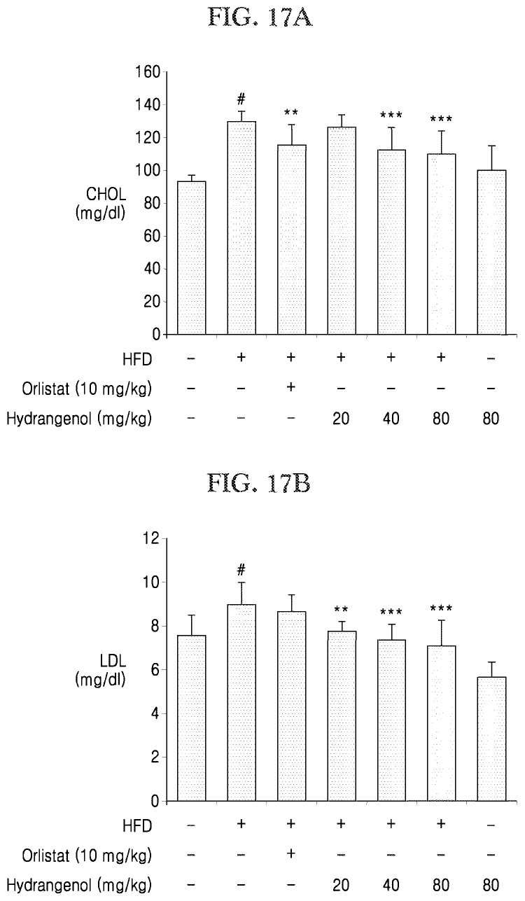

As a result, as shown in Table 4 and FIG. 17, it was confirmed that the HG-administered group showed reduction in the total cholesterol and LDL, but no significant difference in the triglyceride and HDL levels. These results indicate that HG has the effects of improving bad blood lipid levels caused by obesity by reducing total cholesterol and LDL while not affecting triglyceride and HDL levels.

FIG. 17A shows a graph showing cholesterol levels when HG was administered; and FIG. 17B shows a graph showing LDL levels when HG was administered.

TABLE 4

| | | | HFD + | HFD + HG | HFD + HG | HFD + HG | HG |
|---|---|---|---|---|---|---|---|
| | CON | HFD | Orlistat | 20 mg/kg | 40 mg/kg | 80 mg/kg | 80 mg/kg |
| CHOL(mg/dl) | 93.00 ± 3.80 | 129.75 ± 5.93 | 115.63 ± 11.87 | 126.78 ± 6.63 | 112.56 ± 13.64 | 110.14 ± 13.90 | 96.20 ± 18.64 |
| LDL(mg/dl) | 7.63 ± 0.92 | 9.00 ± 1.00 | 8.60 ± 0.84 | 7.78 ± 0.44 | 7.38 ± 0.74 | 7.13 ± 1.13 | 5.70 ± 0.67 |
| HDL(mg/dl) | 69.82 ± 8.89 | 84.73 ± 4.65 | 83.70 ± 5.96 | 80.78 ± 2.91 | 71.89 ± 6.33 | 71.20 ± 8.16 | 66.40 ± 13.75 |
| TG(mg/dl) | 46.09 ± 7.08 | 57.27 ± 9.55 | 76.40 ± 9.47 | 74.63 ± 10.68 | 58.89 ± 11.92 | 63.78 ± 9.94 | 80.10 ± 14.16 |

### 4-6 Analysis of Expression of Energy Metabolism-Related Proteins by HG

AMPK is activated when energy in hepatocytes decreases to maintain energy homeostasis in the liver, thereby inhibiting synthesis of fat and cholesterol, and conversely, promoting fatty acid oxidation. Therefore, to confirm whether administration of HG increased AMPK expression, an experiment was performed.

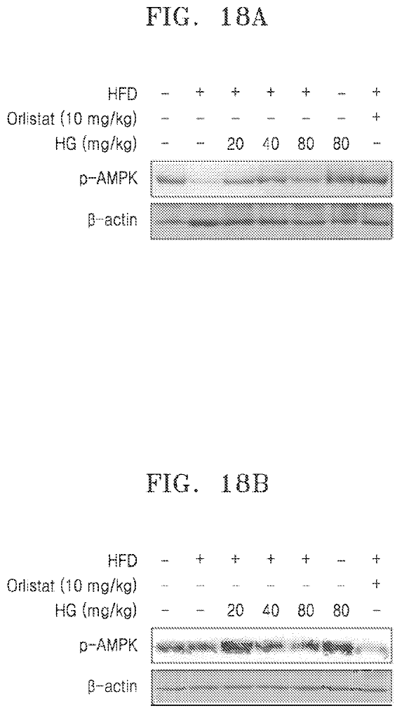

Specifically, adipose and liver tissues were mixed with a protein extraction solution, homogenized using a tissue homogenizer, and then centrifuged at 4° C., 15,000 rpm for 30 minutes to obtain a supernatant, and then a standard curve was created using the Bradford method to quantify protein. 6× sample buffer was added to 30 μg of the protein, followed by heating in a water bath for 5 minutes. Electrophoresis was performed using a 10% SDS-PAGE gel, and immunoblotting was performed on a PVDF membrane for 1 hour and 20 minutes. After blocking for 1 hour with TBST buffer solution containing 5% (w/v) skim milk, anti-p-AMPK antibody was diluted to 1:1000 and reacted at 4° C. for 18 hours. After washing three times with the TBST buffer solution for 10 minutes, the membrane was reacted with a peroxidase-conjugated secondary antibody for 2 hours at room temperature. After washing three times for 10 minutes with the TBST buffer solution, a hyper film was color-developed and developed using an enhanced chemiluminescence kit (Amersham Life Sciences, Amersham, U.K.) to examine the change of AMPK phosphorylation in each control group and experimental group. As a result, in the HG-treated group, the amount of phosphorylated AMPK protein increased (FIG. 18).

These results indicate that HG has the AMPK phosphorylation-inducing effect which is critical in the anti-obesity effect.

FIG. 18A shows results of examining expression of p-AMPK protein in the adipose tissue when HG was administered; and FIG. 18B shows results of examining expression of p-AMPK protein in the liver when HG was administered.

### Preparation Example 1: Preparation of Tablet

A tablet was prepared by mixing components of Table 5 below with hydrangenol and tableting the mixture according to a common method of preparing a tablet.

TABLE 5

| Name of raw material | Unit weight (mg) |
|---|---|
| Hydrangenol | 10.0006 |
| Silicon dioxide | 15.3000 |
| Magnesium stearate | 10.8000 |
| Crystalline cellulose | 799.4945 |
| Hydroxypropyl methylcellulose | 29.0700 |
| Calcium carboxymethyl cellulose | 27.0000 |
| Glycerin fatty acid ester | 0.6930 |
| Titanium dioxide | 1.4697 |
| Monascus red | 4.4082 |
| Caramel pigment powder | 1.7640 |

### Preparation Example 2: Preparation of Capsule

A capsule was prepared by mixing components of Table 6 below with hydrangenol and packing a gelatin capsule with the mixture according to a common method of preparing a capsule.

TABLE 6

| Name of raw material | Unit weight (mg) |
|---|---|
| Hydrangenol | 2 |
| Vitamin E | 2.25 |
| Vitamin C | 2.25 |
| Palm oil | 0.5 |
| Hydrogenated vegetable oil | 2 |
| Yellow wax | 1 |
| Lecithin | 2.25 |
| Soft capsule filling solution | 387.75 |

### Preparation Example 3: Preparation of Jelly

A jelly was prepared by mixing components of Table 7 below with hydrangenol and packing a three-sided pack with the mixture according to a common method of preparing a jelly suitable for preference.

TABLE 7

| Name of raw material | Unit weight (mg) |
|---|---|
| Hydrangenol | 0.0030 |
| Food gel | 0.3600 |
| Carrageenann | 0.0600 |
| Calcium lactate | 0.1000 |
| Sodium citrate | 0.0600 |
| Complex Scutellaria | 0.0200 |
| baicalensis extract | |
| Enzyme-treated stevia | 0.0440 |
| Fructooligosaccharide | 5.0000 |
| solution | |
| Red grape concentrate | 2.4000 |
| Purified water | 13.9560 |

### Preparation Example 4: Preparation of Nourishing Cream

A nourishing cream was prepared using hydrangenol according to a composition of Table 8 below according to a common method.

TABLE 8

| Raw material | Content (%) |
|---|---|
| Hydrangenol | 0.01 |
| Sitosterol | 4.0 |
| Polyglyceryl-2 oleate 3.0 | 3.0 |
| Ceteareth-4 | 2.0 |
| Cholesterol | 3.0 |
| Dicetyl phosphate | 0.4 |
| Concentrated glycerin | 5.0 |
| Sunflower oil | 22.0 |
| Carboxyvinyl polymer | 0.5 |
| Triethanolamine | 0.5 |
| Preservative | Trace amount |
| Flavoring | Trace amount |
| Purified water | Residual quantity |

The above composition ratio is generally formulated as a Preparation Example by mixing suitable ingredients, but the mixing ratio and raw materials may be arbitrarily changed, as needed.

Since the samples of the present disclosure are stable under the experimental conditions of all Preparation Examples, there is no problem in stability of the formulations.

1. A method of improving or treating obesity, the method comprising administering an effective amount of hydrangenol or a pharmaceutically acceptable salt thereof to an individual in need thereof.

2. The method of claim 1, wherein the hydrangenol is represented by the following Formula 1:

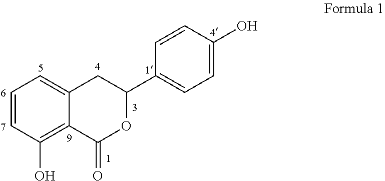 [SMILES: O=C1OC(c2ccc(O)cc2)Cc2cccc(O)c21]

3. The method of claim 1, wherein the hydrangenol or the pharmaceutically acceptable salt thereof inhibits fat formation or reduces body fat.

4. The method of claim 1, wherein the hydrangenol or the pharmaceutically acceptable salt thereof reduces an expression level of peroxisome proliferator-activated receptor gamma γ (PPAR) or reduces adipogenic factors.

5. The method of claim 1, wherein the hydrangenol or the pharmaceutically acceptable salt thereof reduces total cholesterol and low density lipoprotein (LDL) in blood.

6. A method of improving or treating a metabolic disease, the method comprising administering an effective amount of hydrangenol or a pharmaceutically acceptable salt thereof to an individual in need thereof.

7. The method of claim 6, wherein the metabolic disease is hyperlipidemia, hypercholesterolemia, diabetes, or dyslipidemia.
